(19) United States
(12) Patent Application Publication
(10) Pub. No.: US 2011/0245703 A1
(43) Pub. Date: Oct. 6, 2011

(54) SYSTEM AND METHOD PROVIDING BIOFEEDBACK FOR TREATMENT OF MENOPAUSAL AND PERIMENOPAUSAL SYMPTOMS

(75) Inventor: Stephen B. CORN, Sharon, MA (US)

(73) Assignee: ENGINEERED VIGILANCE, LLC, Sharon, MA (US)

(21) Appl. No.: 13/078,856

(22) Filed: Apr. 1, 2011

Related U.S. Application Data
(60) Provisional application No. 61/320,209, filed on Apr. 1, 2010.

(51) Int. Cl. A61B 5/08

(52) U.S. Cl. 600/529

(57) ABSTRACT

An apparatus, system and method for non-contact monitoring of respiratory and/or cardiac functions that is used to provide appropriate biofeedback to a monitored subject in order treat menopausal and perimenopausal symptoms is discussed. Respiratory and/or cardiac waveforms are generated based on monitored physiologic functions. The generated waveforms are analyzed and compared to a target waveform resulting from breathing techniques designed to address menopausal and perimenopausal symptoms. Appropriate biofeedback is generated for the monitored subject based on the analysis of the generated waveform.

### RELATED APPLICATION

The present application is related to and claims the benefit of U.S. Provisional Patent Application No. 61/320,209, filed Apr. 1, 2010, entitled “System and Method Providing Biofeedback for Treatment of Menopausal and Perimenopausal Symptoms”, the contents of which are incorporated herein by reference it its entirety.

### BACKGROUND

According to the 2000 census, about two million women turn 50 every year and fifty-one is the average age of onset of menopause. 85% of women in the US experience some form of hot flashes or night sweats during perimenopause and in the year or two following the beginning of menopause. Furthermore, up to 50% of women continue to report symptoms for years after the onset of menopause. Additionally, it has recently been shown that the use of Tamoxifen for breast cancer prophylaxis is increasing the prevalence of those suffering from hot flashes, due to hot flashes being a side-effect of the therapy. The prophylaxis use of Tamoxifen includes its use by premenopausal women who otherwise would not be suffering from hot flashes.

Hot flashes associated with menopause and perimenopuase (or the use of Tamoxifen or other drugs) can be extremely disruptive to a woman's life regardless of whether the hot flashes are intermittent or near-continuous. The hot flashes not only cause embarrassment and distraction, but can also sometimes be quite frightening. Women affected by hot flashes have described the experience as being trapped in a small room with the heat turned up to the highest level.

Stress is now being recognized by conventional medicine as a major cause of hot flashes and night sweats. A recent study done on over 400 menopausal women showed a direct correlation between anxiety and the severity and frequency of the women's hot flashes. Similarly, a National Institute of Health (NIH) study demonstrated that deep-paced breathing and relaxation exercises done through the day significantly decrease the frequency and severity of flushing symptoms, thus further supporting the precept that stress is a major trigger of menopausal symptoms.

### BRIEF SUMMARY

Relaxation techniques, biofeedback and self-focus on physiologic processes can positively impact general well-being, mitigate the effects of chronic stress and anxiety, decrease morbidity and mortality and treat menopausal and perimenopausal symptoms. The embodiments of the present invention provide a mechanism for performing non-contact monitoring of an individual's physiologic (e.g.: respiratory and/or cardiac) functions that is used to provide appropriate biofeedback to a monitored subject in order to reduce stress and anxiety and treat menopausal and perimenopausal symptoms. Respiratory and/or cardiac waveforms are generated based on the monitored physiologic functions. The generated waveforms may be analyzed and compared to a target waveform for the monitored subject. The target waveform may be determined based on a variety of factors including age, occupation and personal and family medical history. Appropriate biofeedback is generated for the monitored subject based on the analysis of the generated waveform. The biofeedback may take a variety of forms including numerical data and waveform displays and may be accompanied by visual, audible, aromatic and tactile feedback, including vibratory feedback. The biofeedback and other forms of feedback are designed so that the user consciously or unconsciously can control respiration to approach or maintain the target waveform.

In one embodiment a biofeedback system for treating menopausal and perimenopausal symptoms includes a respiratory waveform detection module. The respiratory waveform detection module performs non-contact monitoring of a subject to detect respiratory motion and programmatically generate a waveform based on the detected respiratory motion. The biofeedback system also includes a computing device-implemented analysis module that programmatically analyzes the generated waveform based upon pre-determined criteria. The analysis compares the generated waveform to a programmatically selected target waveform. The biofeedback system further includes a biofeedback module providing biofeedback to the subject to assist the subject in obtaining or maintaining the target waveform. The biofeedback is selected based on a result of the analyzing of the generated waveform.

In another embodiment, an integrated biofeedback apparatus for treating menopausal and perimenopausal symptoms includes a respiratory waveform detection module. The respiratory waveform detection module performs non-contact monitoring of a subject to detect respiratory motion and programmatically generate a waveform based on the detected respiratory motion. The biofeedback apparatus also includes a computing device-implemented analysis module that programmatically analyzes the generated waveform based upon pre-determined criteria. The analysis compares the generated waveform to a programmatically selected target waveform. The biofeedback apparatus further includes a biofeedback module providing biofeedback to the subject to assist the subject in obtaining or maintaining the target waveform. The biofeedback is selected based on a result of the analyzing of the generated waveform. A display surface is used to provide the biofeedback in the form of a display of the generated waveform and a target waveform.

In one embodiment, a method for providing biofeedback to treat menopausal and perimenopausal symptoms includes performing non-contact monitoring of a subject to detect respiratory motion of a subject. The method generates programmatically a waveform based on the detected respiratory motion and analyzes with a computing device the generated waveform based upon pre-determined criteria. The analyzing compares the generated waveform to a programmatically selected target waveform. The method also provides biofeedback to the subject to assist the subject in obtaining or maintaining the target waveform with the biofeedback being based on a result of the analyzing of the generated waveform.

### BRIEF DESCRIPTION OF THE DRAWINGS

The accompanying drawings, which are incorporated in and constitute a part of this specification, illustrate one or more embodiments of the invention and, together with the description, explain the invention. In the drawings:

FIG. 1 depicts an exemplary environment suitable for practicing embodiments of the present invention;

FIG. 2 depicts an exemplary integrated biofeedback and monitoring apparatus;

FIG. 3 depicts an exemplary sequence of steps performed by an embodiment of the present invention to provide biofeedback to a monitored subject;

FIG. 4 depicts an exemplary sequence of steps performed by an embodiment of the present invention to determine whether an intermediate waveform should be provided as biofeedback to a monitored subject;

FIG. 5 depicts user-customizable music and image settings on a mobile phone; and

FIG. 6 depicts an exemplary waveform that may be displayed to a user as the user to assist the user in learning breathing techniques for dealing with menopausal and perimenopausal symptoms.

### DETAILED DESCRIPTION

Research has demonstrated that consciously altering respiration can lead to a change in heart rate (typically a decrease) resulting in a positive impact on physiologic function, and may decrease resting catecholamine levels. Previous attempts at controlling physiologic processes have attempted to have a subject replicate an idealized breathing waveform in an effort to achieve desirable changes in heart rate and catecholamine levels. For example, some forms of meditation encourage their practitioners to practice specific breathing patterns so as to achieve a resting or meditative state consistent with an idealized waveform. Additionally, a number of previous techniques have attempted to control physiologic functions in a test subject by providing biofeedback information (information about physiological processes) to the test subject regarding cardiac function so as to achieve health benefits. One example of this approach is having the participant contact a pad measuring a heart rate. The subject then tries to alter his or her breathing so that a change in heart rate is observed. This system requires direct contact to a sensor and only indirectly sees the effects of a change in respiration, which is the primary function consciously being altered.

A major drawback to previous conventional techniques providing biofeedback is that these conventional techniques require contact monitoring which create an artificial condition. The fact that the monitored subject is aware of the monitoring is counter-productive in that it tends to increase stress levels. Additionally, the use of contact monitoring also makes it difficult to monitor subjects during the course of regular (leisure and occupational) activities.

In contrast to these previous techniques, the embodiments of the present invention utilize a non-contact monitoring system to monitor physiologic functions of a subject which are analyzed to provide biofeedback in real-time that is directed to reducing stress and anxiety for the monitored subject. Non-contact measurement of breathing parameters (e.g.: rate, rhythm, amplitude, pauses, inspiratory to expiratory ratio, breathing frequency variability), and/or cardiac parameters (e.g.: rate, rhythm, amplitude) and/or body movements are utilized to allow a subject to track, visualize and respond to these physiologic signals. The subject achieves an altered physiologic state that positively impacts morbidity and mortality. For example, it has been demonstrated that by consciously altering one's breathing, heart rate can be reduced, the reduction of which leads to a more favorable physiologic condition, in which catecholamine levels and oxygen consumption are reduced.

The biofeedback provided by the embodiments of the present invention may include the display of breathing, cardiac and body movement waveforms and numerical data which can be viewed and analyzed by a monitored subject in real-time so that the subject can alter his or her breathing so as to to positively impact well-being. The biofeedback may also be accompanied by additional visual, audible, aromatic and tactile feedback to create a relaxing environment for the monitored subject. The biofeedback may be used by the subject to achieve a target respiratory or cardiac waveform to facilitate reductions in stress and anxiety.

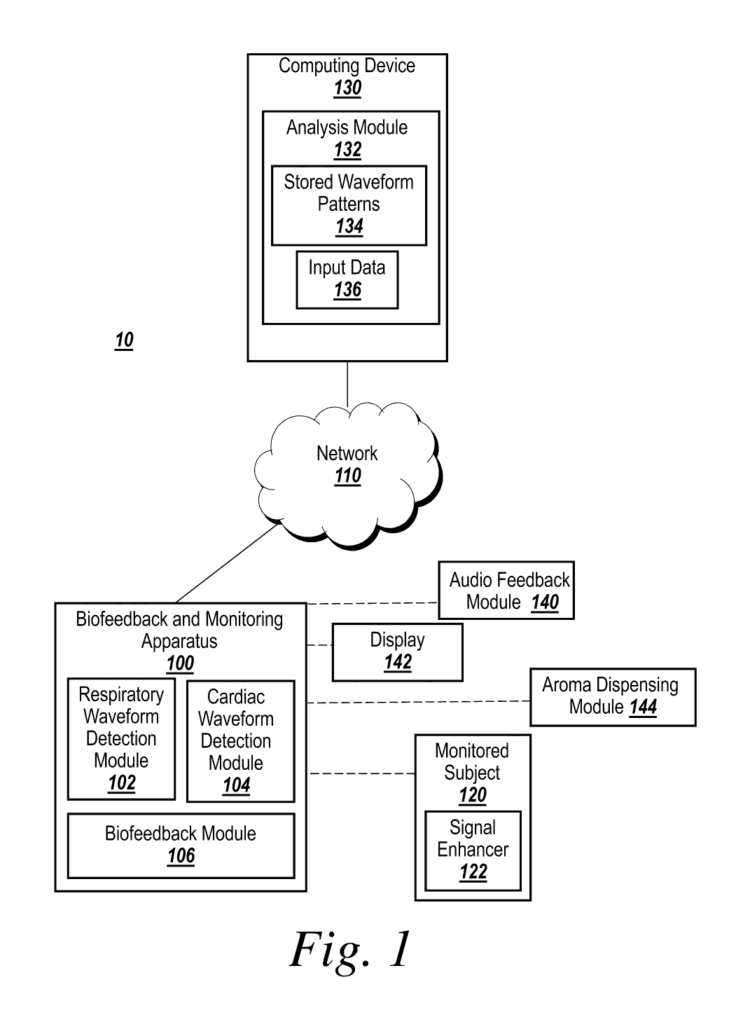

FIG. 1 depicts an exemplary environment suitable for practicing embodiments of the present invention. Biofeedback and monitoring system 10 may include biofeedback and monitoring apparatus 100 that is used to monitor physiological factors for a monitored subject 120. Biofeedback and monitoring apparatus 100 may include a respiratory waveform detection module 102. Respiratory waveform detection module 102 is used to perform non-contact respiratory monitoring of monitored subject 102 and to generate a waveform representing the monitored respiratory process. A number of different techniques to perform the non-contact monitoring may be used and are described in greater detail below.

Biofeedback and monitoring apparatus 100 may also include a cardiac waveform detection module 104. Cardiac waveform detection module 104 is used to perform non-contact cardiac monitoring of monitored subject 102 and to generate a waveform representing the monitored cardiac process. A number of different techniques to perform the non-contact cardiac monitoring may be used and are described in greater detail below.

Once a waveform representing the monitored respiratory or cardiac function has been generated, biofeedback and monitoring system 10 analyzes the generated waveform to determine whether the current monitored physiologic process is optimal. In one embodiment, the generated waveform is programmatically analyzed by a software analysis module 132 executing on a computing device 130. Computing device 130 may take many forms, including but not limited to a personal computer, workstation, server, network computer, quantum computer, optical computer, bio computer, Internet appliance, mobile phone or other mobile device such as a smartphone, a pager, a tablet computer, or other form of computing device equipped with a processor and able to execute analysis module 132. Computing device 130 may be electronic and may include a Central Processing Unit (CPU), memory, storage, input control, modem, network interface, etc. The CPU may control each component of computing device 130 to provide an environment suitable for executing analysis module 132. The memory on computing device 130 temporarily stores instructions and data and provides them to the CPU so that the CPU can operate the computing device 130.

Optionally, computing device 130 may include multiple CPUs for executing software loaded in memory and other programs for controlling system hardware. Each of the CPUs can be a single or a multiple core processor. The code loaded in the memory may run in a virtualized environment, such as in a Virtual Machine (VM). Multiple VMs may be resident on a single processor. Also, part of the code could be run in hardware, for example, by configuring a field programmable gate array (FPGA), using an application specific instruction set processor (ASIP) or creating an application specific integrated circuit (ASIC).

Input control for the computing device 130 may interface with a keyboard, mouse, microphone, camera, such as a web camera, or other input devices such as a 3D mouse, space mouse, multipoint touchpad, accelerometer-based device, gyroscope-based device, etc. Computing device 130 may receive, through the input control, input data relevant for calculating target waveforms for monitored subject 120. Optionally, computing device 130 may display data relevant to the generated waveform on a display as part of the analysis process.

In one embodiment, biofeedback and monitoring apparatus 100 communicates with computing device 130 over a network 110. Network 110 may be the Internet, intranet, LAN (Local Area Network), WAN (Wide Area Network), MAN (Metropolitan Area Network), wireless network or some other type of network over which biofeedback and monitoring apparatus 100 and computing device 130 can communicate. Although depicted as a separate device in FIG. 1, it should also be appreciated that computing device 130 may be part of an integrated apparatus with biofeedback and monitoring apparatus 100.

Analysis module 132 analyzes the generated waveform produced by biofeedback and monitoring apparatus 100. The analysis may be performed using pre-determined criteria. The generated waveform may be compared against stored waveform patterns 134 to determine whether the current generated waveform represents an optimal waveform for the monitored physiologic process. The selection of the comparison waveform from the stored waveform patterns may utilize previous input data 136 that includes information regarding the monitored subject such as personal medical information (e.g. sex, height, weight, age, family history of various diseases, etc. and occupational information). The stored waveforms may be based on stored data from previously monitored subjects. Alternatively, the stored waveform data may be accumulated from monitoring individuals while the individuals perform deep breathing, relaxation breathing or paced breathing exercises as described further herein. Based on available data, the analysis module 132 selects either a customized target waveform or a default waveform for comparison to the generated waveform.

As noted, in one embodiment, the analysis of the generated waveform may be a programmatic process that occurs in a completely automated fashion. In an alternate embodiment, the process may also involve human input in reviewing the selection of the target waveform prior to completion of the analysis. In one embodiment, all of the analysis decisions are saved for future study in order to continually refine the stored waveform patterns 134.

The results of the analysis performed by the analysis module 132 are provided to biofeedback module 106. It should be appreciated that in some embodiments, the functionality attributed to the analysis module 132, the biofeedback module 106 and other modules discussed herein may be combined into one or more modules or split into additional modules without departing from the scope of the present invention. Depending upon the results of the analysis, biofeedback module 106 may take a number of actions. If the monitored subject is already exhibiting a waveform for the monitored physiologic process consistent with the desired target waveform, biofeedback module 106 may provide limited or no biofeedback. Alternatively, the biofeedback module 106 may provide alternative sensory feedback designed to create an environment conducive to maintaining the current respiratory or cardiac function. For example, biofeedback module 106 may provide only audible feedback such as music via audio module 140 or aromatic feedback via aroma dispensing module 144 designed to maintain the status quo. As another option, in such a situation, biofeedback module 106 may provide no feedback at all as the monitored subject has already achieved a desired waveform.

If the results of the analysis performed by analysis module 132 show a discrepancy between the generated waveform (representing the monitored physiologic process) and the target waveform selected by the analysis module, the biofeedback module may provide biofeedback such as providing biofeedback via a display 142 visible to monitored subject 120. The biofeedback may include graphical representations of the generated waveform and target waveform and numerical values representing current physiological measurements. In one embodiment, the two waveforms may be superimposed over each other. Biofeedback module 106 may also provide numerical data such as current respiratory and/or cardiac rates and visual instructions to monitored subject 120 suggesting the monitored subject take a particular action (e.g.: begin a slow breathing exercise to attempt to control respiration rate) that will move the subject towards the target waveform.

In one embodiment, the analysis module 132 may report a significant discrepancy between the generated waveform of the monitored physiological process and the target waveform that exceeds a pre-determined parameter. In such a circumstance, biofeedback module 106 may provide an intermediate waveform to monitored subject 120 rather than the target waveform in an attempt to incrementally adjust the monitored physiological process. The intermediate waveform in such a situation may represent a more attainable goal to monitored subject 120 and its use may prevent the monitored subject from becoming alarmed (which is counter-productive) over the size of the difference between the generated and target waveforms. Biofeedback module 106 may provide a number of intermediate waveforms as appropriate for the monitored subject to attempt to replicate as part of the biofeedback in order to incrementally move the monitored subject towards his or her target waveform. The embodiments of the present invention thus provide the ability to adjust real-time non-contact biofeedback based on the subject's actual response to the intervention. This method is consistent with the movement to personalized medicine where interventions are made specific to a user, not just a population.

The non-contact monitoring system may use radiated energy (e.g.: ultrasonic, radio frequency, infrared, laser, etc.) to identify cardiac and respiratory waveforms in monitored subjects. In some embodiments, the monitoring system illuminates a subject in radiated energy and then detect the reflected radiated energy caused by respiratory and/or cardiac functions. The detected reflections are used to plot a two-dimensional waveform. The waveforms represent the rise and fall of a detected signal (the reflected energy) over time and are indicative of the small movements of a subject's chest, abdomen and/or other anatomical sites that are associated with respiratory and/or cardiac function. Different implementations of the monitoring system use different forms of radiated energy (e.g.: laser, radio frequency or ultrasonic energy) to capture breathing and cardiac waveforms for analysis. Following analysis, appropriate biofeedback is provided to the monitored subject.

One example of a suitable non-contact monitoring system that may be leveraged in conjunction with the embodiments of the present invention is described in U.S. Pat. No. 6,062,216 ('216 patent). As described in the '216 patent, a respiratory monitor may employ either ultrasonic or laser monitoring of an individual's breathing or cardiac function by measuring changes in body position with respect to time. The device continuously and without the need for contact, monitors the individual's breathing and cardiac function (and analyzes the measured waveform and identifies respiratory rate, apneic pauses, and obstructive breathing) and heart rate and rhythm, and body movements. The '216 patent (the contents of which are hereby incorporated by reference) describes a monitoring system using laser energy or ultrasonic energy to monitor respiratory function so as to detect sleep apnea but may be adapted to perform the respiratory and cardiac monitoring described herein.

It should be appreciated that although the monitoring system of the '216 patent has been cited as an exemplary monitoring system which may be used in the present invention, other non-invasive monitoring systems utilizing radiated energy to detect respiratory and/or cardiac waveforms may also be used within the scope of the present invention. For example, in another embodiment, an ultrasound probe connected to a smartphone functions as a biofeedback and monitoring apparatus of the present invention. The ultrasound probe may be used to project radiated ultrasonic energy towards a monitored subject. Ultrasonic energy is a vibration at a frequency above the range of human hearing, in other words usually in a range above 20 kHz. The ultrasound probe illuminates a monitored subject with ultrasonic waves and tracks how long the returned waves take to reflect back to the probe. Measurements may be taken over 100× a second. The gathered data may be communicated to the smartphone in a number of different ways. For example, the ultrasound probe may communicate via an earphone jack, microphone port, through a multi-pin port or dock connector, through a USB port, via BLUETOOTH (if the probe is equipped with its own power source such as a battery) or some other type of communication interface. The gathered data is analyzed by the analysis module. Since sound travels at known speed, Time of Flight measurement may be used to create waveforms and analyze chest movements to identify cardiac and respiratory function.

In another embodiment, the respiratory waveform detection module 102 and the cardiac waveform detection module 104 may use a shaped transducer in the monitoring system to radiate a preferably continuous beam of ultrasound in the 25 kHz to 500 kHz range to illuminate a subject. A receiving transducer in the monitoring system of the present invention or transducer array develops one or more signals, which shift slightly from the incident frequency due to cardiac or respiratory motion. The signal is then analyzed and plotted to generate a waveform. The generated waveform may then be compared against an appropriate benchmark such as a target waveform. Appropriate adjustments are made by the monitoring system to account for the distance between the monitoring system and the subject as well as any environmental factors affecting the detection of the reflected energy.

In one embodiment, the monitoring system may use laser detection means as described in the '216 patent in place of ultrasonic energy. In such a case a laser illuminates the subject in a beam of light of a selected wavelength and the reflected energy which varies based on respiratory and/or cardiac movements is traced so as to generate a waveform. Infrared, radio frequency or other wavelengths that are highly distinct from the spectral range of other light sources surrounding the subject may be selected so as to ease the detection of the reflected energy.

In one embodiment the biofeedback module 106 displays the breathing waveform derived from the non-contact measurement of breathing. Of note, the breathing waveform can be captured through clothes and does not need a specific window to receive the necessary information to generate a breathing or cardiac waveform. However, in one embodiment, a signal enhancer 122 may be utilized to augment the reflected signal. This may be in the form of a “relaxation patch” worn by the participant.

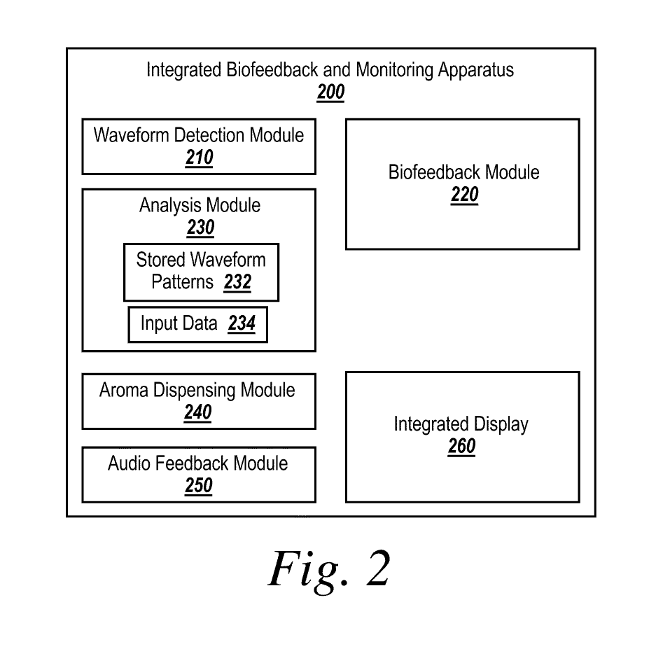

In one embodiment, the biofeedback and monitoring system described herein may be provided as an integrated biofeedback and monitoring apparatus rather than as separate components in multiple devices. FIG. 2 depicts an exemplary integrated biofeedback apparatus 200 that includes most or all of the components of the biofeedback and monitoring system described in FIG. 1. The integrated biofeedback apparatus 200 may include one or more waveform detection modules 210 such as respiratory waveform detection modules and cardiac waveform detection modules. The integrated biofeedback apparatus 200 may also include biofeedback module 220 and analysis module 230. It will be appreciated that biofeedback module 220 and analysis module 230 may be combined into a single module or split into additional modules without departing from the scope of the present invention.

Analysis module 230 may include stored waveform patterns 232 and stored input data 234 specific to a monitored subject. In one embodiment, integrated biofeedback apparatus 200 may also include an aroma dispensing module 240 an audio module 250 for providing aromatic and audio feedback and an integrated display module 260 utilized to provide visual biofeedback to a monitored subject in the manner described herein. In other embodiments, integrated biofeedback apparatus 200 may contain some but not all of the modules 240, 250 and 260 used to provide feedback and biofeedback. The aroma dispensing module 240 may include one or more stored scents that are designed to be soothing when inhaled and that are released into the monitored subject's environment upon a signal received from the biofeedback module 206. In one embodiment, the aroma dispensing module 240 may be separate from, but in communication with, the integrated biofeedback apparatus 200.

In one exemplary embodiment, the integrated biofeedback apparatus 200 may be provided via a portable device such as a mobile phone, tablet computing device or laptop. For example, the mobile phone, tablet computing device or laptop may be equipped with an ultrasound probe that is part of the device or connected via USB and that is used to perform ultrasound monitoring. Similarly, the biofeedback module and analysis modules described herein may be pre-installed or downloaded to the device. The phone, tablet computing device or laptop display and speakers may be used to provide visual biofeedback and audio feedback respectively.

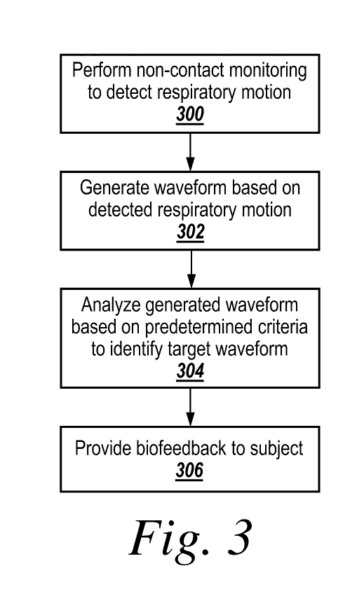

FIG. 3 depicts an exemplary sequence of steps performed by an embodiment of the present invention to provide biofeedback to a monitored subject. The sequence may begin by providing non-contact monitoring as described herein of a subject to detect respiratory motion (step 300). Of note, the subject may or may not be aware of the monitoring. In one embodiment, the subject is informed of the beginning of the monitoring and attempts to perform breathing exercises to enter a relaxed state. In another embodiment, background monitoring may be conducted as part of a normal background process. For example, the monitoring could be performed continually at work and the subject only notified and provided with biofeedback in the event that sub-optimal physiological factors were detected. The embodiments of the present invention thus provide for baseline respiratory and other parameters when affirmative relaxation exercises are not being conducted. This gathering of information outside of a relaxation exercise and response thereto represents a departure from conventional biofeedback techniques that rely on intervening and changing physiologic parameters based on monitoring when the subject is conscious and focused on the monitoring. The embodiments of the present invention are thus beneficial to reducing background stress and anxiety leading to long-term alterations in breathing, heart rate and the like which are important for morbidity and mortality reduction. For example, during the period when relaxation exercises are not being actively being employed, should physiologic parameters be found to be out of range, soothing music, aromatic or visual therapy can be automatically instituted. The time interval at which the monitoring is conducted can be user set, set by a healthcare professional, or set based on monitored responses from the subject.

A waveform is generated as a result of the monitoring process (step 302) and analyzed based on pre-determined criteria to identify a target waveform for the monitored subject (step 304). Appropriate biofeedback is then provided to the monitored subject based on the analysis (step 306). For example, the subject's breathing, and or heart rate and body movement waveforms or numerical data may be displayed. The subject can then alter his or her breathing to attempt to achieve idealized target waveforms, which can be superimposed and displayed on a display surface with the subject's actual waveforms.

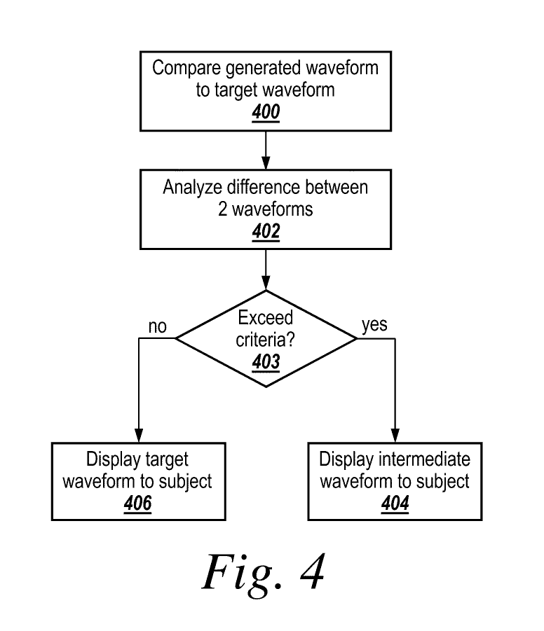

As noted above, in one embodiment the biofeedback module determines that the monitored subject should be presented with an intermediate waveform. FIG. 4 depicts an exemplary sequence of steps performed by an embodiment of the present invention to determine whether an intermediate waveform should be provided as biofeedback to a monitored subject. The sequence begins when the generated waveform from the monitoring of the subject is compared to a target waveform (step 400). The differences between the two waveforms are analyzed (step 402) and a determination is reached as to whether or not the difference between the two waveforms exceeds pre-determined criteria (step 403). If the difference does exceed the criteria, one or more intermediate waveforms between the generated waveform and the target waveform are presented (in sequence if necessary) to the subject as part of the biofeedback (step 404). If the difference does not exceed the criteria, the target waveform is presented to the subject as usual (step 406).

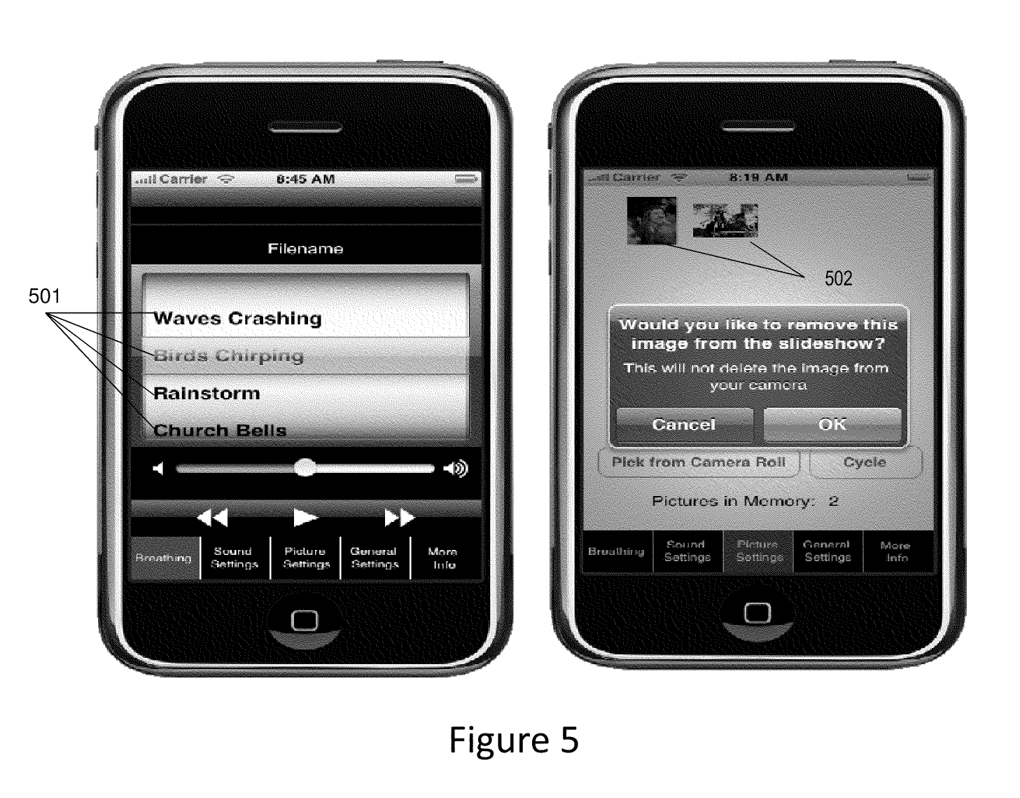

Given the ability to capture and display physiologic waveforms without any direct contact to the subject, the embodiments of the present invention are well suited to also apply known therapies to facilitate relaxation and stress reduction goals. That is, getting the participant to reduce his or her breathing rate can be coupled with visual, aromatic or auditory cues to further enhance this beneficial effect. In an additional aspect of the present invention, the system may make the subject aware of their own breathing without attempting to make the subject alter their breathing, since breathing awareness, even without alteration, has been shown to have health benefits relating to morbidity and mortality. In such an embodiment, visual, aromatic, audible and tactile feedback may be delivered to the subject with or without a waveform being displayed. Music or visual images can be displayed in response to both positive and negative physiologic responses on the system. In one embodiment, the form of the music, visual images or other biofeedback being presented to a user may customizable by the user. For example, in one embodiment, the user may adjust settings on a mobile phone being utilized as the biofeedback apparatus of the present invention. For example, as depicted in FIG. 5, the user may select the particular audio selections 501 or images 502 presented in a slideshow that are generated by the phone in response to monitored physiological responses.

Music or visual images can be displayed in response to both positive and negative physiologic responses on the system. In one embodiment, the form of the music, visual images or other biofeedback being presented to a user may be customizable by a user or the monitored subject. For example, in one embodiment, the user may adjust settings on a mobile phone being utilized as the biofeedback and monitoring apparatus of the present invention. The monitored subject or other user may select the particular audio selections or images presented in a slideshow that are generated by the phone in response to monitored physiological responses. Thus, in the case where an integrated biofeedback and monitoring apparatus such as an IPHONE or IPAD is being deployed, a user may customize audio feedback by selecting songs from their own ITUNES library and/or customize visual content by selecting static or dynamic images such as pictures, slideshows or video from their own content stored on, or accessible from, the portable device acting as the integrated biofeedback and monitoring apparatus.

For example a user or subject may select birds chirping as a background sound during a breathing exercise, delete a particular image from a slideshow or select a photo cycle time that will be applied to visual biofeedback. The user may also select video content as part of the customization process and may select a background image for a display surface. It should be appreciated that the customization options discussed herein are exemplary and are not intended to be an exhaustive list and that other types of customization are also within the scope of the present invention.

As noted, the monitored information and analysis decisions may be stored. The ability to store the monitored information allows an objective response to therapy, provides storage for medical records and may be utilized for third party reimbursement. Further, having objective and permanent records of responses to therapy adds to the attractiveness of the technique to medical professionals and leads to better compliance with a relaxation regime by the monitored subjects.

It should be understood that other physiologic parameters, (video, audio, etc) could be incorporated to add robustness to the proposed system. Further, though breathing, cardiac and body movement are optimally derived through non-contact means to prevent the creation of an overly artificial environment, a contact monitoring system may also be used to perform monitoring of a subject.

In attempting to assist a monitored subject in achieving a target waveform of the present invention, biofeedback-based breathing exercises may be utilized. As a result of a search in recent years for safer and more effective treatment modalities for dealing with menopausal symptoms, it has been demonstrated that deep breathing techniques can shorten hot flashes and make them milder in up to 50% of participants. Deep breathing, Relaxation Breathing, and Paced Respiration all refer to a method used to reduce stress. The US Government's National Institute of Aging recommends these breathing techniques to reduce the incidence and severity of hot flashes. The breathing techniques are believed to trigger the parasympathetic nervous system, which calms one down and helps regulate temperature.

The breathing techniques involve breathing in (inhaling) deeply and breathing out (exhaling) at an even pace. This is done in a comfortable position for several minutes for several minutes. The technique involves slowly breathing in through the nose. With a hand on the stomach right below the ribs, the participant should first feel the stomach pushing the hand out, and then the chest should fill. Slowly exhaling through the mouth, first letting the lungs empty and then feeling the stomach sink back. One can do this almost anywhere and is advised to do so several times during the day, whenever feeling stressed. The exercises are also recommended if one is feeling a hot flash beginning.

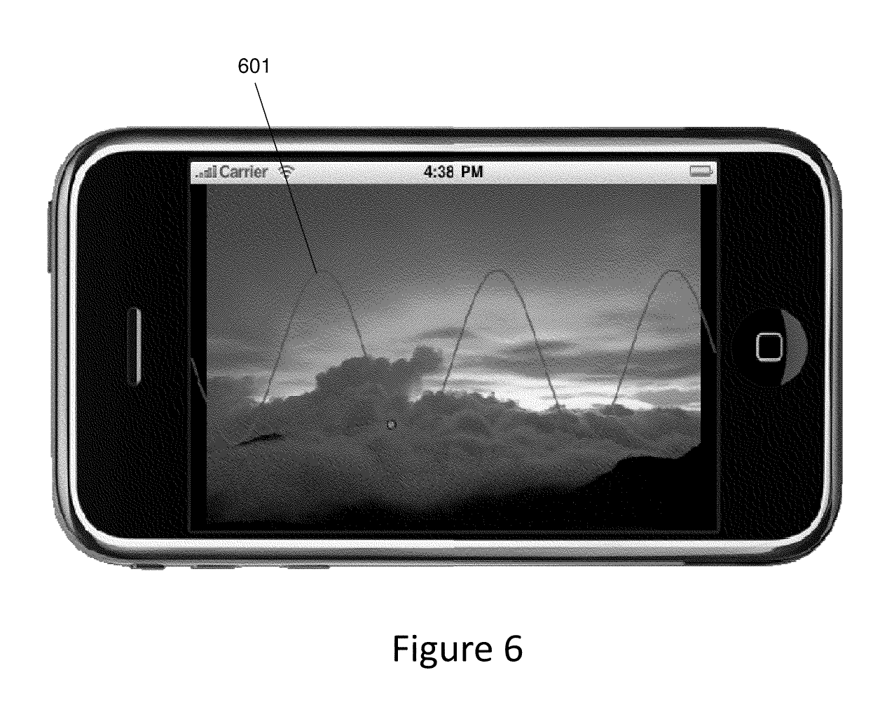

If one practices deep breathing techniques, like yoga breathing or Pilates breathing, before one actually needs them, they will come more naturally during the stress and embarrassment of a hot flash episode. Training ahead of time is believed to be important in decreasing the incidence and severity of hot flashes. To this end, the embodiments of the present invention can be used to monitor a woman's respiration and provide biofeedback designed to help the woman learn the breathing techniques of Deep breathing, Relaxation Breathing, and Paced Respiration. The biofeedback provided by the embodiments of the present invention can indicate to the woman whether a desired breathing technique is being performed properly or improperly. For example, FIG. 6 depicts an exemplary waveform 601 that may be displayed to a user to assist a user in learning breathing techniques for dealing with menopausal and perimenopausal symptoms. While the monitoring and biofeedback can occur during menopausal/perimenopausal episodes, more ideally, the monitoring takes place during non-episode times so that the woman can learn the techniques needed to deal with the symptoms before experiencing the hot flash or other symptom.

One aspect of the present invention also provides a social networking component whereby women who are being monitored with the biofeedback system as described herein can share experiences and techniques for dealing with their menopausal and perimenopausal symptoms. A remote server may be used to connect the women into a virtual community if they wish to share their experiences of using the techniques learned from the biofeedback system of the present invention or in dealing with their medical condition.

In another aspect of the present invention, women who are being monitored utilizing the biofeedback system as described herein may be offered a ‘tip of the day’ by the biofeedback system for dealing with their menopausal and perimenopausal symptoms. The tip may register as an alert or a background item in a display being presented to the woman. As an alert, the tip may be timed to when the user is scheduled to begin her breathing exercises or each iteration of her exercises during a day. Additionally, the “tip” may also be accompanied by targeted advertisements. The ‘tip of the day’ may also be an optional feature which may be declined by a user.

The present invention may be provided as one or more computer-readable programs embodied on or in one or more non-transitory physical mediums. The mediums may be a floppy disk, a hard disk, a compact disc, a digital versatile disc, a flash memory card, a PROM, an MRAM, a RAM, a ROM, or a magnetic tape. In general, the computer-readable programs may be implemented in any programming language. Some examples of languages that can be used include C, C++, C#, Python, FLASH, JavaScript, or Java. The software programs may be stored on, or in, one or more mediums as object code. Hardware acceleration may be used and all or a portion of the code may run on a FPGA, an Application Specific Integrated Processor (ASIP), or an Application Specific Integrated Circuit (ASIC). The code may run in a virtualized environment such as in a virtual machine. Multiple virtual machines running the code may be resident on a single processor.

Since certain changes may be made without departing from the scope of the present invention, it is intended that all matter contained in the above description or shown in the accompanying drawings be interpreted as illustrative and not in a literal sense. Practitioners of the art will realize that the sequence of steps and architectures depicted in the figures may be altered without departing from the scope of the present invention and that the illustrations contained herein are singular examples of a multitude of possible depictions of the present invention.

The foregoing description of example embodiments of the invention provides illustration and description, but is not intended to be exhaustive or to limit the invention to the precise form disclosed. Modifications and variations are possible in light of the above teachings or may be acquired from practice of the invention. For example, while a series of acts has been described with regard to FIGS. 3-4, the order of the acts may be modified in other implementations consistent with the principles of the invention. Further, non-dependent acts may be performed in parallel.

In addition, implementations consistent with principles of the invention can be implemented using devices and configurations other than those illustrated in the figures and described in the specification without departing from the spirit of the invention. Devices and/or components may be added and/or removed from the implementations of FIGS. 1-2, 5 and 6 depending on specific deployments and/or applications.

We claim:

1. A biofeedback system for treating menopausal and perimenopausal symptoms, comprising:
a respiratory waveform detection module, the respiratory waveform detection module performing non-contact monitoring of a subject to detect respiratory motion and programmatically generating a waveform based on the detected respiratory motion;
a computing device-implemented analysis module programmatically analyzing the generated waveform based upon pre-determined criteria, the analyzing comparing the generated waveform to a programmatically selected target waveform; and
a biofeedback module providing biofeedback to the subject to assist the subject in obtaining or maintaining the target waveform, the biofeedback selected based on a result of the analyzing of the generated waveform.

2. The system of claim 1, further comprising:
a display, the display used to provide the biofeedback in the form of a display of the generated waveform and a target waveform.

3. The system of claim 2 wherein the display of the generated waveform is accompanied by instructions for the subject to alter a rate of breathing so as to attain the target waveform.

4. The system of claim 1, further comprising:
a display, the display used to provide the biofeedback in the form of a display of an intermediate waveform that represents a waveform between the generated waveform and the target waveform.

5. The system of claim 1 wherein the biofeedback module provides the intermediate waveform based on the analyzing indicating a difference between the target waveform and the generated waveform that is greater than a pre-determined criteria.

6. The system of claim 1, further comprising:
an aroma dispensing module that is used as an adjunct or to provide feedback in the form of aromas detectable by the subject.

7. The system of claim 1, further comprising:
an auditory module that is used as an adjunct or to provide feedback in the form of audio transmissions detectable by the subject.

8. The system of claim 1 wherein the analysis module uses personalized data concerning an occupation or physical condition of the subject in determining the target waveform.

9. The system of claim 1 wherein the target waveform was initially generated from data collected by monitoring subjects performing breathing techniques designed to address menopausal and perimenopausal symptoms.

10. An integrated biofeedback apparatus for treating menopausal and perimenopausal symptoms, comprising:
a respiratory waveform detection module, the respiratory waveform detection module performing non-contact monitoring of a subject to detect respiratory motion and generating a waveform based on the detected respiratory motion;
a computing-device implemented analysis module programmatically analyzing the generated waveform based upon pre-determined criteria, the analyzing comparing the generated waveform to a programmatically selected target waveform;
a biofeedback module providing biofeedback to the subject to assist the subject in obtaining or maintaining the target waveform, the biofeedback based on a result of the analyzing of the generated waveform; and
a display surface, the display surface used to provide the biofeedback in the form of a display of the generated waveform and a target waveform.

11. The apparatus of claim 10 wherein the target waveform was initially generated from data collected by monitoring subjects performing breathing techniques designed to address menopausal and perimenopausal symptoms.

12. The apparatus of claim 10, further comprising:
an auditory module that is used to provide feedback in the form of audio transmissions detectable by the subject.

13. The apparatus of claim 10, further comprising:
an aroma dispensing module that is used to provide feedback in the form of aromas detectable by the subject.

14. The apparatus of claim 10 wherein the respiratory waveform detection module monitors the subject using ultrasound.

15. The apparatus of claim 10 wherein the respiratory waveform detection module monitors the subject using laser detection means.

16. The apparatus of claim 10 wherein the respiratory waveform detection module monitors the subject using infrared or radio frequency transmissions.

17. A method for providing biofeedback to treat menopausal and perimenopausal symptoms, comprising:
performing non-contact monitoring of a subject to detect respiratory motion of a subject;
generating programmatically a waveform based on the detected respiratory motion;
analyzing with a computing device the generated waveform based upon pre-determined criteria, the analyzing comparing the generated waveform to a programmatically selected target waveform; and
providing biofeedback to the subject to assist the subject in obtaining or maintaining the target waveform, the biofeedback based on a result of the analyzing of the generated waveform.

18. The method of claim 17 wherein the target waveform was initially generated from data collected by monitoring subjects performing breathing techniques designed to address menopausal and perimenopausal symptoms.

19. The method of claim 17, further comprising:
providing the biofeedback in the form of a display of the generated waveform and a target waveform.

20. The method of claim 19, further comprising:
conveying instructions for the subject to decrease a rate of breathing so as to attain the target waveform.

21. The method of claim 17, further comprising:
providing the biofeedback in the form of a display of an intermediate waveform that represents a waveform between the generated waveform and the target waveform.

22. The method of claim 21 wherein the intermediate waveform is displayed based on the analyzing indicating a difference between the target waveform and the generated waveform that is greater than a pre-determined criteria.

23. The method of claim 17, further comprising:
providing feedback in the form of an aroma detectable by the subject.

24. The method of claim 17, further comprising:
providing feedback in the form of an audio or visual transmission detectable by the subject.

25. The method of claim 17 wherein the analyzing uses personalized data concerning an occupation or physical condition of the subject in determining the target waveform.

26. The method of claim 17 wherein the biofeedback is provided prior to the subject becoming aware of the monitoring.

27. A physical computer-readable medium holding computer-executable instructions for providing biofeedback to treat menopausal and perimenopausal symptoms, the instructions when executed causing one or more devices to:
perform non-contact monitoring of a subject to detect respiratory motion of a subject;
generate programmatically a waveform based on the detected respiratory motion;
analyze the generated waveform based upon pre-determined criteria, the analyzing comparing the generated waveform to a programmatically selected target waveform; and
provide biofeedback to the subject to assist the subject in obtaining or maintaining the target waveform, the biofeedback based on a result of the analyzing of the generated waveform.

28. The medium of claim 27 wherein the target waveform was initially generated from data collected by monitoring subjects performing breathing techniques designed to address menopausal and perimenopausal symptoms.

29. The medium of claim 27 wherein the execution of the instructions further causes the one or more devices to:
provide the biofeedback in the form of a display of the generated waveform and a target waveform.

30. The medium of claim 29 wherein the execution of the instructions further causes the one or more devices to:
convey instructions for the subject to decrease a rate of breathing so as to attain the target waveform.

31. The medium of claim 27 wherein the execution of the instructions further causes the one or more devices to:
provide the biofeedback in the form of a display of an intermediate waveform that represents a waveform between the generated waveform and the target waveform.

32. The medium of claim 27 wherein the execution of the instructions further causes the one or more devices to:
provide feedback in the form of an aroma detectable by the subject.

33. The medium of claim 27 wherein the execution of the instructions further causes the one or more devices to:
provide feedback in the form of an audio transmission detectable by the subject.

34. The medium of claim 27 wherein the analyzing uses personalized data concerning an occupation or physical condition of the subject in determining the target waveform.
